(19) United States
(12) Patent Application Publication
(10) Pub. No.: US 2026/0157851 A1
(43) Pub. Date: Jun. 11, 2026

(54) ADJUSTABLE ANNULOPLASTY RING

(71) Applicant: EDWARDS LIFESCIENCES CORPORATION, Irvine, CA (US)

(72) Inventors: Rodolfo Rodriguez, San Luis Obispo, CA (US); Pui Tong Ho, Ladera Ranch, CA (US); Allison Nicole Rabin, Costa Mesa, CA (US); Thomas Howell Rickards, Santa Ana, CA (US); Max Sotnick, San Jose, CA (US); Graham Isaak, Portland, OR (US)

(21) Appl. No.: 18/974,022

(22) Filed: Dec. 9, 2024

(51) Int. Cl. A61F 2/24

(52) U.S. Cl. CPC A61F 2/2448; A61F 2230/0095; A61F 2250/0004; A61F 2250/0058

(57) ABSTRACT

Cybernetically enhanced adjustable annuloplasty rings and deployment systems. The annuloplasty rings are size-adjustable after implant, and each may have a hollow circumference with a cinching element extending therethrough whose length is controlled remotely. A size adjustment mechanism housed within a junction housing is powered by a piezo-electric motor that has no ferromagnetic components. The motor may connect to the junction housing via a flexible cable, and be located outside the heart.

### TECHNICAL FIELD

The present disclosure relates generally to annuloplasty rings, and in particular to a cybernetic, remotely adjustable mitral annuloplasty ring.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Various surgical techniques may be used to repair a diseased or damaged valve. A commonly used repair technique effective in treating incompetence is annuloplasty, which often involves reshaping or remodeling the annulus by attaching a prosthetic annuloplasty repair segment or ring thereto. The procedure is done with the heart stopped and the patient on cardiopulmonary bypass (“on pump”). For instance, the goal of a posterior mitral annulus repair is to bring the posterior mitral leaflet forward toward to the anterior leaflet to improve leaflet coaptation. Annuloplasty rings may be stiff, flexible or semi-rigid, and a “remodeling” annuloplasty ring typically has an inner core that is “generally rigid” or “semi-rigid” in that it will flex to a small extent but resist distortion when subjected to the stress imparted thereon by the mitral valve annulus of an operating human heart.

Currently, during a mitral valve repair procedure, the size of the annuloplasty ring is determined by comparing different sizer templates to the patient's anatomy until the surgeon determines which one looks correct based on, for example, anterior leaflet area or length, intercommissural distance, and so on. However, unlike for an aortic valve replacement, where the goal is to implant the largest valve that will safely fit the patient's anatomy, mitral repair procedures implant a repair device that is somewhat smaller than the annulus to reduce the perimeter, or, more importantly, the anterior-posterior (AP) diameter, of the valve and restore leaflet coaptation. The surgeon must make an “educated guess” as to how much reduction in size is appropriate for any given patient and their specific disease state. If the wrong size repair product is chosen, the result may be a poor outcome manifested by residual mitral regurgitation (MR), insufficient coaptation length, high pressure gradients, or systolic anterior motion (SAM). If any of these conditions are found once the patient is weaned off-pump, the surgeon must make the difficult decision of going back on pump, with its associated morbidity and mortality, or leaving the patient with a sub-optimal repair, and its associated sequalae.

Given the above challenges, it would be desirable to have an annuloplasty device that could be adjusted once the patient was weaned off-pump in order to fine-tune the AP diameter (short axis) or AL-PM diameter (long axis) of the mitral valve in order to correct for small errors in the inherently imprecise sizing process. Such a ring would have the potential to reduce poor mitral valve repair outcomes and the need to go back on-pump in many cases to address them. Once adjustments were made and desired outcome achieved, the deployment system attachments could be disengaged, leaving the patient with a customized annuloplasty device that was tailored to their specific anatomy.

In attempts to vary the shape of the repair device, adjustable annuloplasty devices such as the Cardinal mitral annuloplasty system originally from Valtech Ltd. of Israel, now a part of Edwards Lifesciences of Irvine, CA. The Cardinal system has a semi-rigid annuloplasty ring enabled for ring diameter fine tuning and optimization of leaflet coaptation on a beating heart under real-time echocardiographic guidance. The Cardinal system is disclosed in U.S. Pat. Nos. 8,241,351 and 10,363,136, the entireties which are expressly incorporated herein by reference for all purposes. However, once implanted and adjusted, any adjustment instruments are removed from the body and all implant incisions are closed up, effectively precluding further adjustments.

Proper sizing of an implanted annuloplasty ring is important to maintain effective valve closure and prevent regurgitation during contraction, but is difficult to assess during an open-heart operation. As a result, if improper sizing is determined post-implantation, further surgical intervention is necessary to correct the mitral valve repair. Therefore, the ability to remotely adjust an annuloplasty ring would greatly improve patient outcomes without requiring additional invasive procedures.

Despite past attempts, there is a need for an adjustable annuloplasty ring that may be shaped adjusted after implant and after sealing up any implant incisions.

### SUMMARY

Disclosed here are cybernetically enhanced adjustable annuloplasty rings and deployment systems. The annuloplasty rings are size-adjustable after implant and facilitate delivery and reduce long-term complications. The rings each have a hollow circumference with a cinching element extending therethrough whose length is controlled remotely. The rings are cybernetic in that they incorporate small motors for adjusting their sizes. Cybernetic enhancement is the use of cybernetics and/or technology to improve the functioning and performance of the human body, such as in prosthetic limbs, microchip implants and the like.

In one embodiment, an adjustable annuloplasty ring has a spring coil attached to a housing containing a spool that is driven by a piezoelectric motor to shorten or lengthen a cinch wire around the ring circumference. The piezoelectric motor is attached to a microcontroller that can accurately control the size of the ring. One benefit to piezoelectric motors is avoiding magnetic or ferromagnetic materials, the presence of which preclude MRI scans on the patient.

Another embodiment of the present application is a mitral valve annuloplasty ring with an integrated motor and external controller capable of being adjusted remotely. The integrated motor is a single-rotational piezoelectric motor and housing integrated into the ring, capped leads implanted subcutaneously to connect to an external motor driver, and an external motor controller. The annuloplasty ring is capable of being operated during both off and on-pump procedures. This will allow the ring to be adjusted during follow-up appointments without requiring further surgical intervention.

Another embodiment includes an annuloplasty ring and shape adjustment system, comprising an annuloplasty ring defining a continuous peripheral shape around a central orifice. A size adjustment mechanism housed within a junction housing adjusts the peripheral shape of the annuloplasty ring and is powered by a piezo-electric motor that has no ferromagnetic components.

In the system above, the annuloplasty ring may be shaped for implant at a mitral annulus having a D-shape with a rounded posterior portion opposite a more linear anterior portion, with side segments in between, and the junction housing is located at a midpoint in the anterior portion. The posterior portion and the anterior portion desirably rise up from the side segments such that the annuloplasty ring forms a saddle shape. Also, the side segments may lie in a reference plane P such that the annuloplasty ring is partially planar.

In the system above, the motor may be attached to the junction housing and rotates a central shaft which engages a rotational device within the junction housing to cinch the annuloplasty ring and reduce the peripheral shape. The largest dimension of the motor is preferably no greater than about 6 mm in any dimension. The rotational device may have a shaft with a diametric passage through which an elongated flexible cinching element passes, the cinching element passing circumferentially around the annuloplasty ring such that actuation of the motor shortens or lengthens the cinching element to respectively reduce or enlarge the peripheral shape. Alternatively, the annuloplasty ring has an elongated flexible cinching element connected to wind around the rotational device, and the cinching element passes circumferentially around the annuloplasty ring within a coiled body having opposite ends attached to the junction housing, wherein actuation of the motor shortens or lengthens the cinching element to respectively reduce or enlarge the peripheral shape.

In any of the systems above, the cinching element may be a stainless steel wire or cable. Further, the coiled body may define a lumen through which a hollow compressible filler member extends, the cinching element passing through a lumen in the filler member, and the annuloplasty ring is covered with a tubular fabric cover with a sewing cuff at a peripheral outer edge.

In some cases, the motor produces a torque of about 30-50 N-mm to adjust the peripheral shape of the annuloplasty ring. The system may further include an implanted transducer that converts an externally applied power into electricity, wherein the motor is powered by the implanted transducer when energized externally. Another option is an internal power source that provides sufficient power to a power management and control system and a data communication system but not the motor. There may also be a sensor mounted on the annuloplasty ring and connected to the internal power source.

Any of the systems above may further include an internal power source that provides sufficient power to a power management and control system and a data communication system and a sensor mounted on the annuloplasty ring and connected to the power management and control system. The sensor may be an acoustic or pressure sensor that detects regurgitation. The system sensor may provide feedback to the motor for automatic cinching if it detects regurgitation. The sensor may also cooperate with the power management and control system and a data communication system to collect and send data to an external control system for a physician to decide whether to adjust the ring.

In any of the systems above, the motor may connect to the junction housing via a flexible cable. The annuloplasty ring may be shaped for implant at a mitral annulus with the motor is adapted to be attached outside of the left atrium and the flexible cable has a sufficient length to extend from outside the left atrial wall to the mitral annulus.

A further understanding of the nature and advantages will become apparent by reference to the remaining portions of the specification and drawings.

### BRIEF DESCRIPTION OF THE DRAWINGS

Features and advantages of the present disclosure will become appreciated as the same become better understood with reference to the specification, claims, and appended drawings wherein:

FIG. 1 is an atrial plan view of a mitral valve and leaflets indicating common nomenclature initials for regular anatomical features;

FIG. 2 is a schematic view of the three-dimensional shape of the mitral annulus with several anatomical landmarks indicated;

FIGS. 3A-3E are orthogonal views of an exemplary annuloplasty ring of the present application;

FIG. 4A is a perspective view of an underside of an adjustable annuloplasty ring of the present application, and FIG. 4B is the same view of the annuloplasty ring with an outer cover removed;

FIG. 5A is a perspective view of the adjustable annuloplasty ring attached to a native mitral annulus and prior to size adjustment, and FIG. 5B is the same view after size adjustment to eliminate regurgitation;

FIG. 6 is a perspective view of a size adjustment mechanism of the adjustable annuloplasty ring;

FIG. 7A is a top plan view of the size adjustment mechanism showing an inner spool around which adjustment wire is wrapped, and FIG. 7B is a sectional view through the spool;

FIG. 8 is an exploded perspective view of the primary components of the adjustable annuloplasty ring;

FIG. 9 is a perspective view of the spool mounted for rotation within a housing shown in phantom;

FIG. 10 is a perspective view of the spool;

FIG. 11 is a top plan view of the adjustable annuloplasty ring showing primary dimensions thereof;

FIGS. 12A and 12B are diagrams indicating two different spool diameters and the torque required to overcome a given resistance to rotation;

FIG. 13 is a schematic view of a procedure for adjusting an annuloplasty ring after implant;

FIG. 14 is a schematic view of components used to control adjustment of the annuloplasty ring;

FIG. 15 is a sectional view through a mitral annulus and surrounding anatomical structure showing an alternative embodiment of a remotely adjustable annuloplasty ring of the present application;

FIG. 16 is a radial sectional view through the annuloplasty ring in FIG. 15 showing internal components thereof;

FIG. 17 is an enlargement of a junction housing within which is mounted the size adjustment mechanism for the annuloplasty ring of FIG. 15;

FIG. 18 is a sectional view of an ischemic left ventricle after implantation of a replacement heart valve and remotely adjustable subvalvular structure; and

FIG. 19 is a sectional view of an ischemic left ventricle after implantation of remotely adjustable neochords.

### DETAILED DESCRIPTION

The right ventricle and left ventricle are separated from the right atrium and left atrium, respectively, by the tricuspid valve and mitral valve; e.g., the atrioventricular valves. Though correction of the mitral annulus is the primary focus of the present application, it should be understood that certain characteristics of the annuloplasty rings described herein may equally be used to treat the tricuspid valve, and thus the claims should not be constrained to the mitral ring unless expressly limited.

The term “axis” in reference to the illustrated annuloplasty rings, and other non-circular or non-planar rings, refers to a line generally through the centroid of the ring periphery when viewed in plan view. “Axial” or the direction of the “axis” can also be viewed as being parallel to the average direction of blood flow within the valve orifice and thus within the ring when implanted therein. Stated another way, an implanted mitral ring orients about a central flow axis aligned along an average direction of blood flow through the mitral annulus from the left atrium to the left ventricle. The plan views of the annuloplasty rings illustrated herein are as looking from the atrial side in the direction of blood flow. For the purpose of orientation, therefore, the atrial side of the ring is up and the ventricular side is down.

The annuloplasty ring system included a closed ring as described below, but the adjustable ring system is also applicable open-ring annuloplasty devices, for example, mitral annuloplasty bands/rings and tricuspid annuloplasty rings.

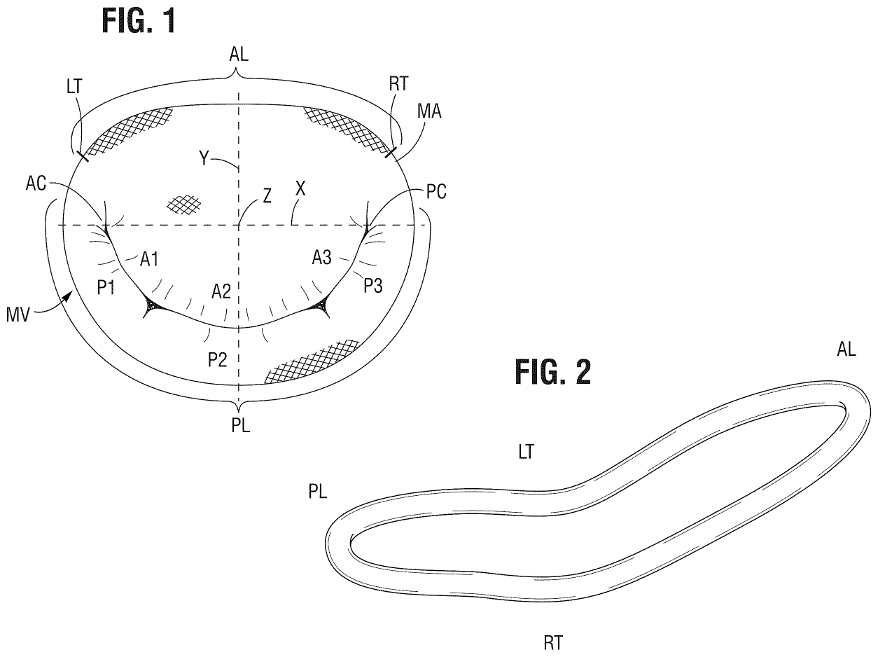

FIG. 1 is a schematic plan view from the atrial side of a mitral valve MV with posterior being down and anterior being up. The mitral valve MV primarily comprises a pair of coapting leaflets—an anterior leaflet AL and a posterior leaflet PL—secured around their outer edges to a fibrous mitral annulus MA. The surrounding mitral annulus MA is often described as D-shaped with a somewhat straighter side adjacent the anterior leaflet AL and a more rounded or convex side adjacent the posterior leaflet PL. The mitral annulus MA is typically viewed as having a major axis X that intersects both the first and third posterior scallops P1 and P3, approximately at the commissures AC, PC, and a minor axis Y that intersects and generally bisects the middle posterior scallop P2. A central flow axis Z is arbitrarily defined at the intersection of the major and minor axes X, Y.

The leaflets are shaped such that the line of coaptation resembles a smile that approximately parallels the posterior aspect of the mitral annulus MA. The anterior leaflet AL spans a smaller peripheral aspect around the mitral annulus MA than the posterior leaflet PL, but the anterior leaflet AL has a convex free edge that extends farther into the orifice defined by the mitral annulus MA. The posterior leaflet PL, on the other hand, has a generally concave free edge. Two commissures—an anterior commissure AC and a posterior commissure PC—generally defined the intersection of the line of coaptation between the two leaflets AL, PL and the mitral annulus MA. The posterior leaflet is divided into three scallops or cusps, sometimes identified as P1, P2, and P3, starting from the anterior commissure AC and continuing in a counterclockwise direction to the posterior commissure PC. Per convention, a major axis of the mitral annulus intersects both the first and third posterior scallops P1 and P3, approximately at the commissures AC, PC, and a minor axis intersects and generally bisects the middle posterior scallop P2. The anterior leaflet also features scallops or regions labeled A1, A2, and A3 as indicated in FIG. 1.

As illustrated, the mitral annulus has a kidney or rounded D-shape around its periphery. The mitral anterior leaflet AL attaches to a somewhat straight anterior fibrous portion of the mitral annulus, which makes up about one-third of the total mitral annulus circumference. The anterior fibrous annulus, the two ends of which are called the fibrous left and right trigones LT, RT, forms part of the central fibrous skeleton of the heart. The arcuate muscular portion of the mitral annulus constitutes the remainder of the mitral annulus, and the posterior leaflet PL attaches thereto. The anterior commissure AC and the posterior commissure PC are located just posterior to each fibrous trigone.

In degenerative mitral regurgitation (DMR), a primary cause for the onset of regurgitation is the dilation of the MV annulus. Typically, the dilation of the annulus is localized in the P2 to P3 region (see FIG. 1). One goal of the present annuloplasty ring is to reshape the annulus to an appropriate size, to support the annulus in the surgically remodeled shape, and to be able to fine-tune the ring shape post-implant.

FIG. 2 is a schematic view of the three-dimensional shape of the mitral annulus with several anatomical landmarks indicated. In particular, the anterior leaflet AL is separated from the posterior leaflet PL by the left and right trigones LT, RT. The three-dimensional shape is somewhat like a saddle, with the trigones LT, RT in a valley and the leaflets AL, PL rising up.

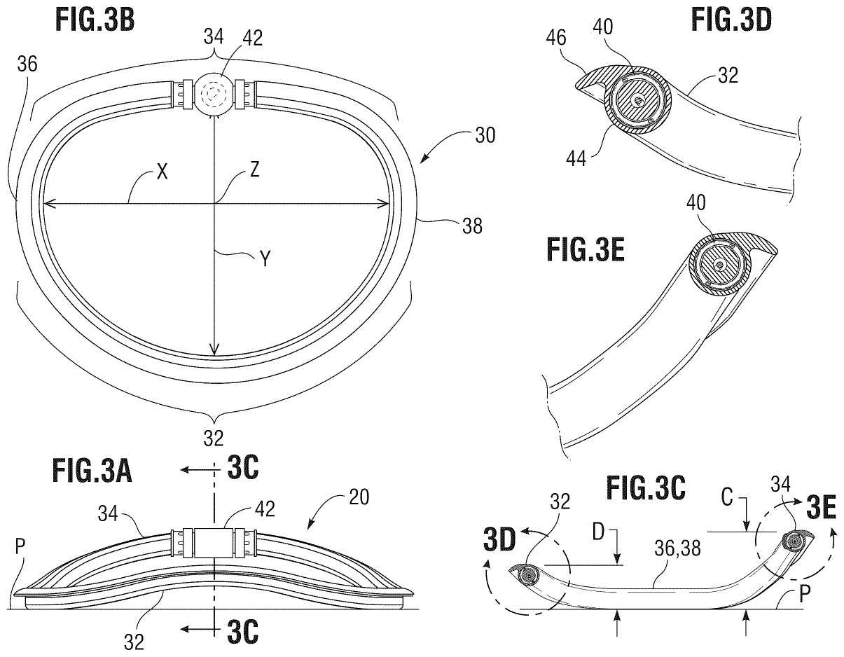

As seen in FIGS. 3A-3C, an exemplary annuloplasty ring 30 has a posterior portion 32 opposite an anterior portion 34, with side segments 36, 38 in between. Some nomenclature has the posterior portion 32 extending roughly around the posterior leaflet PL (FIG. 2), extending between the leaflet commissures AC, PC, but generally the posterior portion 32 is bisected by the minor axis Y and extends at least around the middle posterior scallop P2 of the posterior leaflet PL. A major axis X and a minor axis Y are indicated which, when the annuloplasty ring 30 is implanted, coincide with the same axes of the native mitral annulus, such that blood will flow into the page generally parallel to a flow axis Z through the middle of the ring. The plan view shape of the annuloplasty ring 30 is kidney or rounded D-shaped so as to conform to the peripheral shape of the typical mitral annulus.

The annuloplasty ring 30 may be three-dimensional with an upward bow in the posterior portion 32 as well as an upward bow in the anterior portion 34, as seen in FIGS. 3A and 3C. Preferably the anterior portion 34 bows upward a distance C from a reference plane P more than an upward bow D of the anterior portion 34. The side segments 36, 38 may lie in the reference plane P such that the ring 30 is partially planar with the two opposite upward bows, to form somewhat of a saddle shape so as to better conform to the native mitral annulus, as depicted in FIG. 2. The shape of the ring 30 is similar to that of the Physio II® annuloplasty ring available from Edwards Lifesciences of Irvine, CA.

FIGS. 3D and 3E are enlarged sectional views of the annuloplasty ring 30 as seen in section in FIG. 3C. In a preferred example, the ring construction includes an adjustable inner core 40, as will be described, and surrounded by a suture-permeable interface. In the illustrated example, the inner core 40 is shown as having a flexible coil with an outer covering, though other configurations are possible. As will be explained below, the inner core 40 is adjustable in dimension, including along both primary axes X, Y as well as in total circumference. As will be described, the annuloplasty ring 30 has a size adjustment mechanism 42 located at a midpoint of the anterior portion 34. In the illustrated embodiment, the adjustment mechanism 42 is contained within an outer housing that joins two free ends of the annuloplasty ring 30. It should be understood that the adjustment mechanism 42 could be located at other places around the ring 30, but the middle of the anterior portion 34 tends to be the most stable location after implant, and thus forms a more stable base around the ring.

The suture-permeable interface may include an elastomeric sleeve 44 closely surrounding the core and a fabric outer cover (not shown), for example, a polyethylene terephthalate (PET) fabric cover. In the preferred example the elastomeric sleeve 44, which may be silicone rubber, is generally tubular and molded to have a radially outwardly-extending flange 46 to facilitate suturing of the ring 30 to the mitral annulus. The ring 30 may be secured with sutures, staples, or other such devices to an inside fibrous ledge of the mitral annulus. In a typical procedure, the surgeon anchors an array of sutures through the annulus and then threads them through corresponding locations around the interface on the outside of the ring 30. The ring is parachuted down the suture array to be seated at the annulus before tying off the sutures.

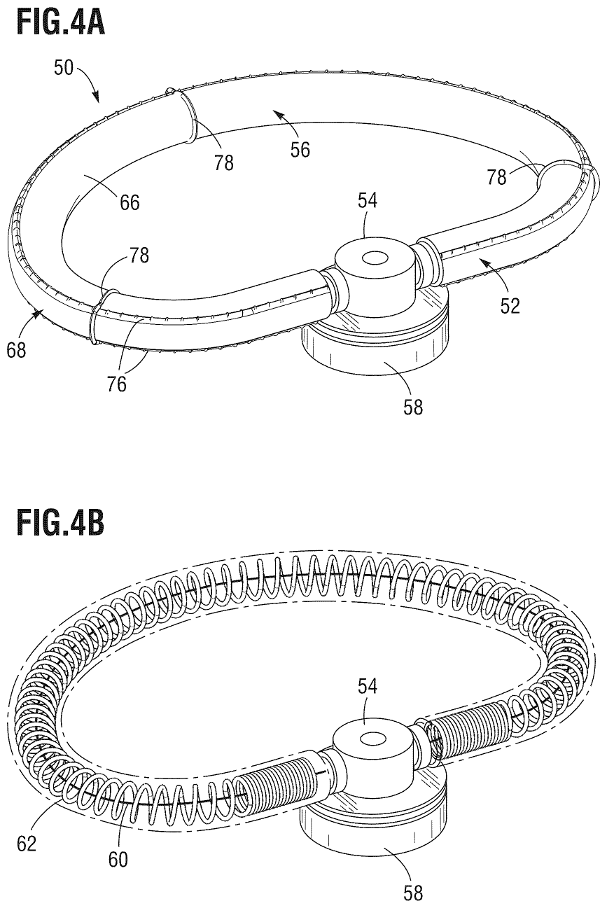

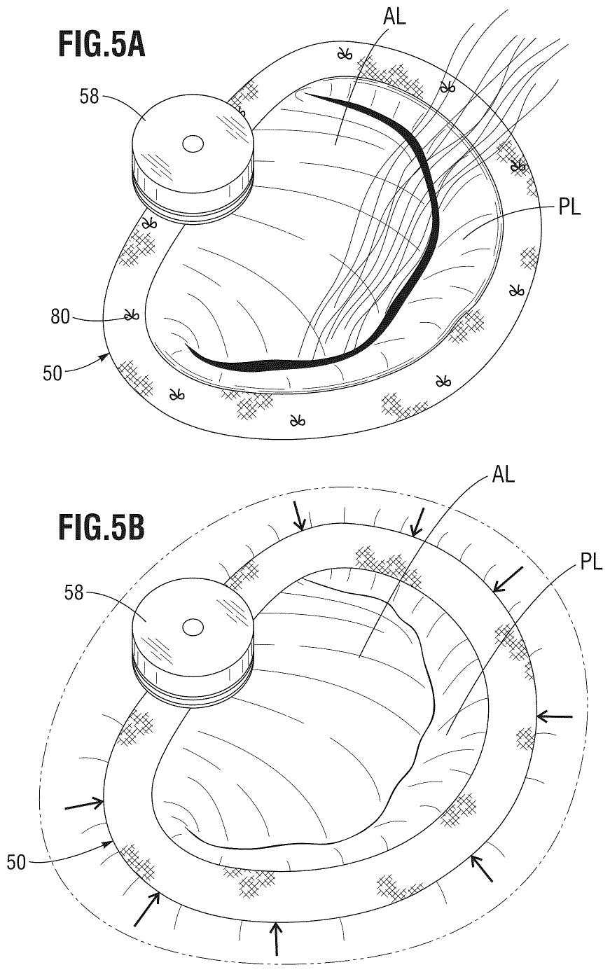

FIG. 4A is a perspective view of an underside of another adjustable annuloplasty ring 50 of the present application, and FIG. 4B is the same view of the annuloplasty ring with an outer cover removed. As mentioned above, the annuloplasty ring defines a rounded D-shape with a straighter anterior portion 52 in which is centered a junction housing 54 opposite a rounded posterior portion 56. In the illustrated embodiment, a generally cylindrical motor 58 attaches to the junction housing 54. As will be explained, the motor 58 rotates a central shaft which engages a spool or other device within the junction housing 54 to adjust the size of the annuloplasty ring 50.

It should be understood that though the annuloplasty ring 50 in these drawings is shown as being planar, it could just as easily be three-dimensional, as seen in FIGS. 3A-3E. It should also be noted that the annuloplasty ring 50 is shown from an underside or outflow side which faces the left ventricle when implanted. The cylindrical motor 58 is desirably as small as possible in diameter, though at this stage miniaturization to a size equivalent to the radio dimension of the annuloplasty ring is not available. Therefore, the motor 58 is positioned on the atrial or inflow side of the ring 50, and the ring is shown upside-down here to better illustrate the components of the adjustment mechanism.

FIG. 4B shows the main internal components of the annuloplasty ring 50, including a cinching element 60 passes circumferentially around the periphery of the ring within a coiled body 62. The spool or other device within the junction housing 54 alters the tension within the cinching element 60 to change the size of the annuloplasty ring 50. Because of the inherent flexibility of the coiled body 62, the ring can be adjusted in a number of ways. One way to adjust the ring as to pull the tension on both free ends of the cinching element 60 that pass into the junction housing 54, which reduces the circumference of the ring 50 in a uniform manner. Alternatively, only one free end of the cinching element 60 may be placed in tension, which reduces the circumference or dimension of that side of the ring more than the other. Likewise, the cinching element 60 may be split into two or more, and anchored at different locations around the coiled body 62, which provides size reduction in different segments of the ring. Those of skill in the art will understand that there are a number of ways to change the size and shape of the annuloplasty ring 50.

Aside from the junction housing 54, the exterior of the annuloplasty ring 50 is covered with a tubular fabric cover 66 with a sewing cuff 68 added to a peripheral outer edge. The fabric cover 66 closely surrounds the coiled body 62 defining a lumen through which a hollow filler member (not shown, see FIGS. 3D, 16) extends; the filler member being made of a soft, compressible elastic material such as silicone. Finally, the cinching element 60 passes around the entire periphery of the annuloplasty ring 50 within a central lumen in the filler member; the cinching element 60 by definition thus extending through the lumen of the coiled body 62. The filler member acts as a cushion between the cinching element 60 and coiled body 62.

The sewing cuff 68 comprises a ring of fabric formed by a single layer that is attached to the fabric cover 66 by a series of stitches 76 along the upper and lower edges (see also the radial sectional view of FIG. 16). In one embodiment, the stitches 76 are colored differently than both the fabric cover 66 and ring of fabric so that the upper and lower edges of the ring of fabric is accentuated. This serves to inform a surgeon where to pass the anchoring sutures-i.e., between the stitches 76 along the upper and lower edges of the ring of fabric. The outer cover 66 further has a plurality of discrete marker bands 78 sewn thereto, at positions indicating the points on the ring corresponding to the fibrous trigones and midpoint of the posterior portion of the mitral annulus. The marker bands 78 help position the ring 50 around the annulus during implantation.

An example ring structure may be adopted from a mechanically adjustable annuloplasty ring, for example, a Mitrafit annuloplasty system (Edwards Lifesciences of Irvine, CA), which includes two main components: a structural meal frame 62, for example, a garter spring or laser-cut tube, and a polyester knit fabric outer layer 66. In an example, the spring can be manufactured using 316L steel, with a decreased spring pitch near the ends integrated to the housing to maintain the desired D shape of the ring during adjustments. The spring stiffness at these solid ends can be about 8073 mN/m, while the stiffness of remaining spring circumference can be about 3975 mN/m. At each end of the ring, there can be a mechanical clasping system that will secure the ring to the housing 54.

FIG. 5A is a perspective view of the adjustable annuloplasty ring 50 attached to a native mitral annulus and prior to size adjustment, and FIG. 5B is the same view after size adjustment to eliminate regurgitation. FIG. 5A shows the implanted annuloplasty ring 50 after the pairs of sutures have been tied off into knots 80 and severed close to the ring. Following the process of anchoring the annuloplasty ring 50 to the mitral annulus MA, the left atrium is closed around any control or power leads with one or more purse string sutures. All other incisions are closed to prevent blood loss, with the size adjustment mechanism remaining such that it may be controlled from outside the body. Subsequently, the patient is weaned off of cardiopulmonary bypass and heart restarted.

Adjustment of the annuloplasty ring 50 to optimize the repair is guided by a visualization technique such as transesophageal echocardiography (TEE) or in rare cases fluoroscopy, mainly focusing on residual mitral regurgitation, degree of leaflet coaptation and the presence of transmitral gradients. For example, FIG. 5A illustrates the mitral valve MV during systole when the anterior leaflet AL and posterior leaflet PL come together or coapt. In this illustration, mitral regurgitation is indicated by the escaping blood flow which can be seen on TEE. This means that the leaflets AL, PL are not coapting, which may be corrected by reducing the size of the annuloplasty ring 50. Even if there is no regurgitation seen, the surgeon may still decide to adjust the ring size in order to obtain a larger surface of coaptation.

FIG. 5B shows the annuloplasty ring 50 being reduced in size from outside the body, or remotely. Cinching the annuloplasty ring 50 in this regard brings the leaflets AL, PL closer together, and also the overall annulus diameter when desired, thus improving coaptation. In the illustration, the mitral valve MV is closed during systole with the leaflets coapting and no regurgitation detected. The annuloplasty ring 50 may be reduced in size by at least 2 mm across the major axis, equivalent to one standard ring size. Further reduction in major axis dimension may be provided up to 4 mm. Following ring size adjustments, any control or power leads may be disengaged from the annuloplasty ring 50 and removed from the body. The purse string suture through the left atrial wall and any other openings are then closed to complete the procedure.

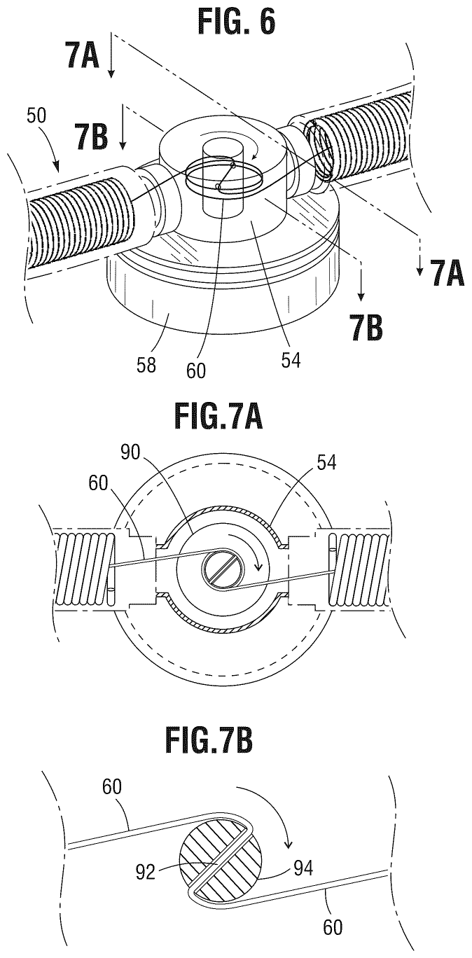

FIG. 6 is a perspective view of an example of a size adjustment mechanism or means of the adjustable annuloplasty ring 50, again looking at the underside thereof for clarity. The illustrated example uses a spool mechanism, around which the cinching element or means, for example, a cable or band, is reversibly windable, for adjusting a size of the ring. Other examples of a size adjustment mechanisms include, for example, a rack-and-pinion mechanism, where a motor drives the pinion gear to adjust one or more racks coupled to or integral with the cinching element; or a hose-clamp-type (worm gear) mechanism, where a motor drives a worm gear engaging a toothed element coupled to or integral with the cinching element. In this example, the two free ends of the annuloplasty ring 50 coupled to the junction housing 54 such that the cinching element 60 can pass through connecting channels and be wrapped around a rotating inner spool 90, seen in FIG. 7A. In one embodiment, the cinching element 60 comprises a stainless steel wire or cable, or other such biocompatible filament that is flexible enough to be wrapped around the spool 90, and also possesses sufficient structural stiffness to resist stretching. FIG. 7B is a somewhat schematic sectional view through the spool 90 in which a central portion 92 of the cinching element 60 extends through a diametric passage through a spool shaft 94. In this way, the cinching element 60 is a continuous filament and clockwise rotation of the spool shaft 94 winds both ends and increases the tension in the cinching element.

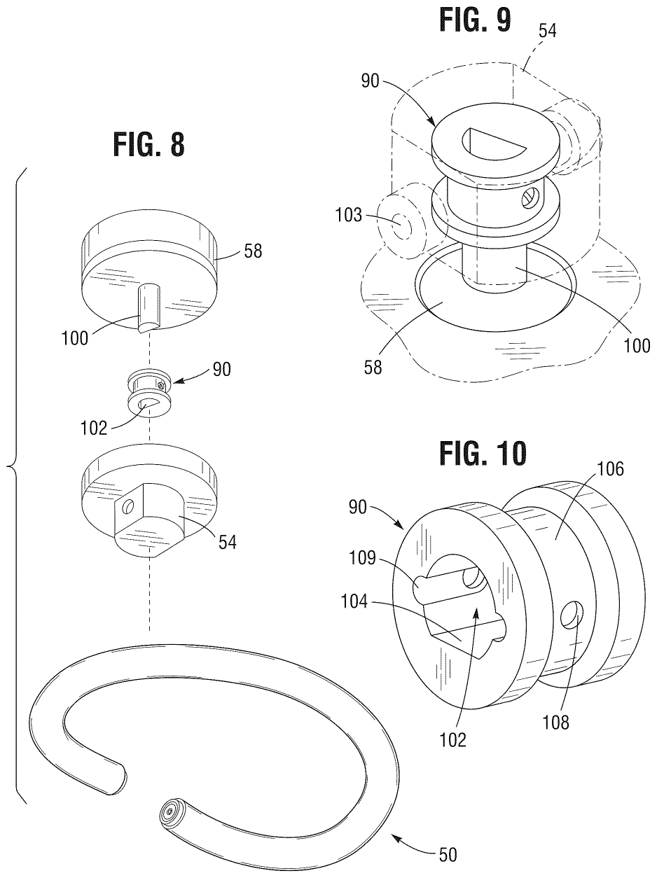

FIG. 8 is an exploded perspective view of the primary components of the adjustable annuloplasty ring 50, showing in series the junction housing 54, rotating spool 90, and drive motor 58. The motor 58 has a driveshaft 100 that may be keyed to fit within a similarly keyed through bore 102 in the spool 90. The spool 90 fits within a cavity in the junction housing 54, as seen in phantom in FIG. 9. The junction housing 54 has lateral ports 103 through which the cinching element 60 extends from both sides. As explained with reference to FIGS. 7A and 7B, the cinching element may extend through a diametric passage in the spool shaft 94, and thus be engaged by the spool 94 increasing or reducing tension therein to change the size of the annuloplasty ring 50.

### Spool

FIG. 10 is a perspective view of the spool 90, which better illustrates the key through bore 102 having a flat keyway 104 on one side thereof. The spool 90 includes a recessed raceway 106 around which the cinching element 60 may be wrapped. In this embodiment, the spool 90 includes a pair of diametrically-opposed openings 108 in the raceway 106 through which the cinching element 90 extends. Short axial passages 109 may be used to route free ends of the cinching element 60, which may be tied off and thus retain the position by the presence of the drive shaft 100. That is, the cinching element 60 may be a continuous wire (e.g., having free ends crimped together), or may have two free ends which are tied off at the spool 90.

The spool 90 connects to the motor with an interference fit around the D-shaped motor shaft 100. There is a single track or raceway 106 to the spool 90 with two holes 108 in the center of the track, each corresponding to opposite ends of the cinching element 60 leading in from either side of the ring, so that adjustments can be made from both sides at once as the spool 90 rotates. The inner radius of the raceway 106, where the cinching element 60 winds around, determines the torque required by the motor: the larger the inner diameter, the larger the torque required to turn the spool 90. As will be explained below.

The spool 90 track is wide enough for multiple loops of wire to be wound during the adjustment. If the ring is over adjusted, the direction of motor rotation can easily be reversed releasing excess wire which relaxes the ring and increases its overall size.

The spool 90 is also responsible for securing the cinching element 60 so it does not slip during adjustment. This is done by threading the wire through the holes on the suture track, which lead to the outside of the spool 90 through the interface between the spool 90 and the shaft. This provides a press fit that holds the wire in place when the spool 90 slides over the shaft, and the wire ends can then be tied outside of the housing for additional resistance to slipping.

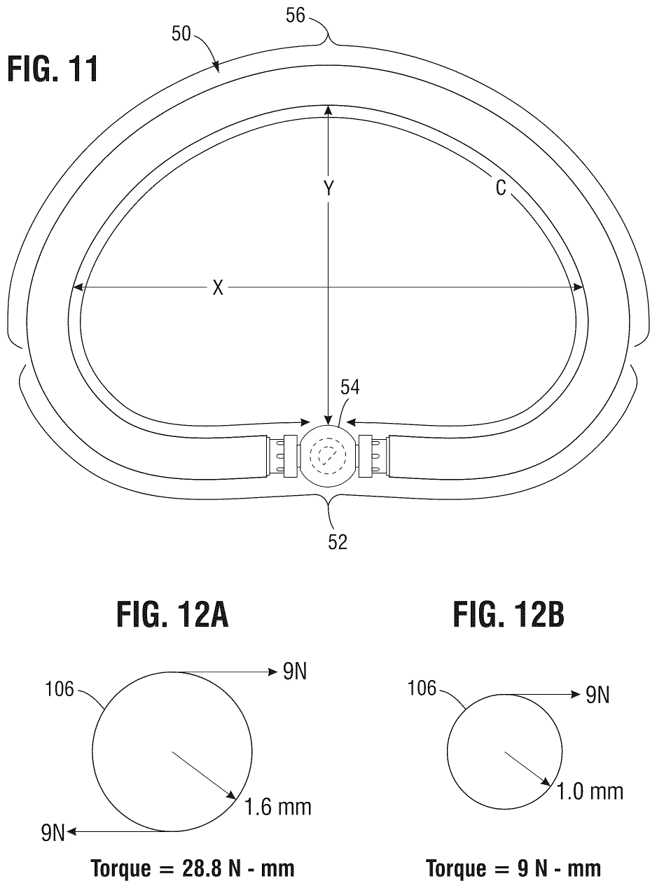

FIG. 11 is a top plan view of the adjustable annuloplasty ring 50 showing primary dimensions thereof. Namely, the major axis has an inner dimension X, the minor axis an inner dimension Y, and an overall inner circumference C. As explained, the junction housing 54 enclosing the adjustment mechanism is desirably located at the center of the anterior portion 52, with the posterior portion 56 shown extending approximately two thirds of the way around the ring 50. Because of the relative stiffness of the anterior aspect of the native annulus, increasing tension on the cinching element 60 will primarily affect the posterior portion 56. Moreover, as seen in FIG. 4B, the flexible coil 62 desirably has a tighter wind adjacent the free ends that connect to the junction housing 54 than around the rest of the circumference. Consequently, tension in a single cinching element 60 that passes through the interior of the entire circumference of the ring 50 primarily reduces the Y dimension of the ring along the minor axis, often referred to as the anterior/posterior, or A/P, dimension. Again, however using multiple cinching elements or anchoring free ends of the cinching elements at various locations around the annuloplasty ring 50 may result in an asymmetric or otherwise customized size adjustments.

Consideration should be given to the diameter of the inner shaft or raceway 106 of the adjustment spool 90 and the resulting torque required for the drive motor 58. FIGS. 12A and 12B are diagrams indicating two different spool raceway diameters and the torque required to overcome a given resistance to rotation. That is, after implant at the annulus a certain amount of force is necessary to constrict the annuloplasty ring 50 and the native annulus. Empirical studies place the force for typical annuloplasty rings at around 9 N. If the diameter of the raceway 106 is about 1.6 mm, as in FIG. 12A, a torque of about 28.8 N-mm from the motor will be required. Alternatively, when the diameter of the raceway 106 is about 1 mm, the required torque is reduced to approximately 9 N-mm. Depending on the amount of miniaturization required of the motor, the torque may be extremely limited and a smaller diameter raceway 106 will be the outcome. However, anticipation of greater motor capacities from improving technology may enable use of larger diameter raceways 106, which also provides faster adjustment of the annuloplasty ring size.

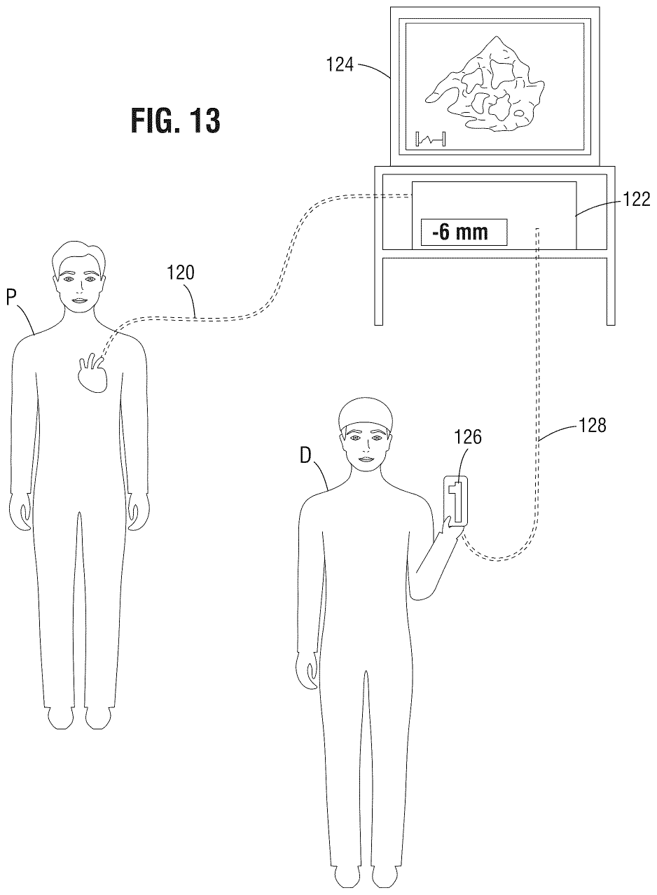

FIG. 13 is a schematic view of a procedure for adjusting an annuloplasty ring after implant. First of all, an adjustable annuloplasty ring 50 is implanted in the patient P as seen in FIG. 5A. The motor 58 contains encoded drive programming responsive to external signals. The signals may be generated by a wireless source, or via a wire connected directly to the motor 58 and extending outside the body. Therefore, lead 120 shown in phantom represents either a physical wire or a wireless signal. Instructions for controlling the size of the annuloplasty ring 50 are generated by a primary supply and control module 122 exterior to the body. A monitor 124 may be connected with the control module 122 to help visualize the size adjustment procedure. A remote-control 126 manipulated by the doctor D provides instructions to the control module 122 via a wireless or hardwired conduit 128. The size adjustment procedure may be accomplished using a physical conduit 120 into the body until the proper size is achieved, after which the wire 120 is removed and all incisions closed up. Subsequently, if further adjustments are required, the motor 58 may also possess wireless capabilities which enable further size adjustments remotely.

### Power Management and Control System

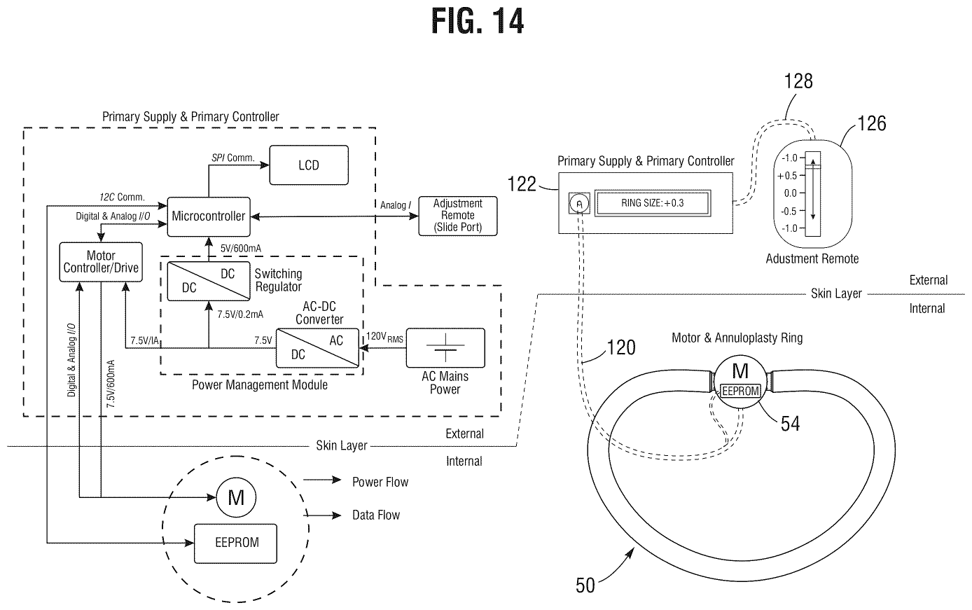

With regards to the electrical circuitry of this project there are two subsystems: the power management circuitry, and the control system circuitry. While these subsystems function logically independent of each other, for the purpose of explanation they will be presented as a relational pair. FIG. 14 shows how both of these subsystems are integrated together in order to operate the motor (among other tasks).

Since the control system includes many different modules, we can further divide the diagram above into the following groups for the purposes of analysis: Power management, Head-control unit, Node-control unit, and the Motor-EEPROM pair.

FIG. 14 is a schematic view of components used to control adjustment of the annuloplasty ring. As in FIG. 13, the communication conduits 120, 128 are shown in phantom to indicate that they may be physically hard-wired, or representative of wireless signals.

One additional aspect may be one or more sensors implanted with the annuloplasty ring. For instance, a suitable sensor may be attached to the ring or within the left atrium LA to detect regurgitation, for example, an acoustic or pressure sensor. The sensor may provide feedback to the motor 58 for automatic cinching if it detects regurgitation, may collect and store data for later retrieval, or may send data to the external control system for a physician to decide whether to adjust the ring. A class of suitable sensors includes a MEMS (micro-electromechanical system) device, which are electromechanical devices fabricated using microprocessor-type manufacturing technologies. Examples of MEMS devices can be small and can have low power requirements.

Another type of sensor that can be incorporated in the system is a temperature sensor, which can detect changes in body temperature. Such a change can indicate a fever, which could be caused by infection.

### Remote Motor Configuration

The above-described example of the adjustable annuloplasty ring configuration involves the drive motor connected directly to the annuloplasty ring. The following example is similar to the previous example except that the motor is spaced from the annuloplasty ring, and coupled thereto with a drive cable or shaft. Such an arrangement can be preferred, for example, where a size of the motor or another component adversely affects blood flow, or where at least one other component of the system, for example, the motor, a power module, a data processing module, a communication module, or a power receiving module, is better situated outside the heart for any other reason.

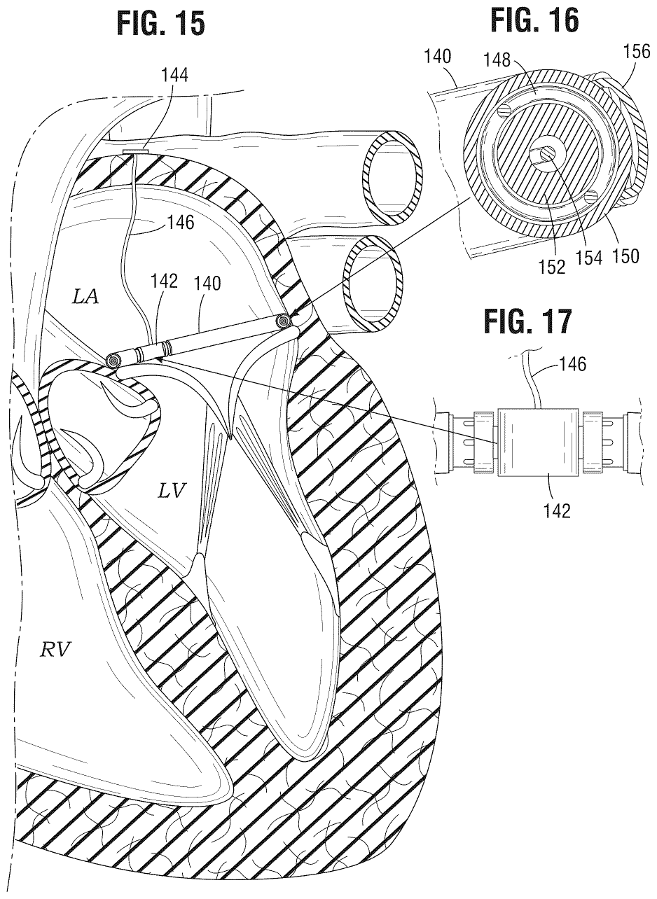

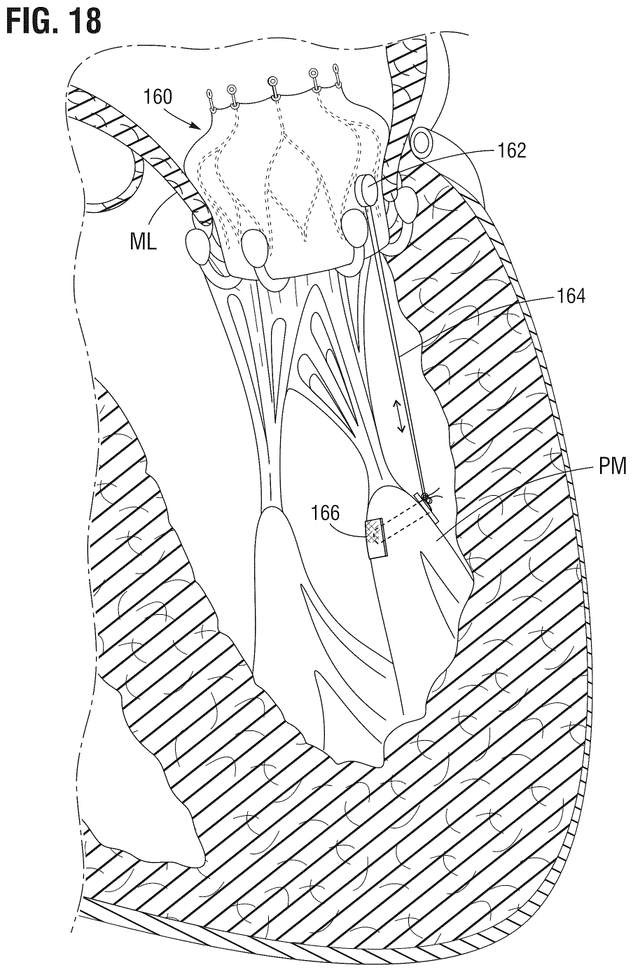

Consequently, FIG. 15 is a sectional view through a mitral annulus and surrounding anatomical structure showing an alternative embodiment of a remotely adjustable annuloplasty ring 140. In this embodiment, an adjustment mechanism within a junction housing 142 such as described previously may be powered by a motor 144 attached outside of the left atrium LA. A flexible drive cable 146 connects the motor 144 to the junction housing 142 (see FIG. 17). The drive cable 146 may have an outer stationery sheath within which a braided or otherwise torque cable rotates within. The torque cable, in turn, is keyed to a rotating element or spool within the junction housing 142. In this way, a larger motor 144 may be utilized than if the motor were connected directly to the ring 140. The location of the motor 144 may vary, but a convenient positioning is just outside the left atrial wall. As before, the motor 144 may be controlled via signals through a physical wire or using wireless technology from outside the body.

FIG. 16 is a radial sectional view through the annuloplasty ring 140 in FIG. 15 showing internal components thereof. As with the embodiment of FIG. 4A/4B, the ring 140 comprises an inner flexible coil 148 surrounded by an outer cover 150. A hollow filler member 152 extends through the lumen of the coil 148, and a cinching element 154 passes through a central through bore within the filler member 152. Finally, an outer sewing cuff 156 may be provided to enhance the ability to connect the annuloplasty ring to the annulus.

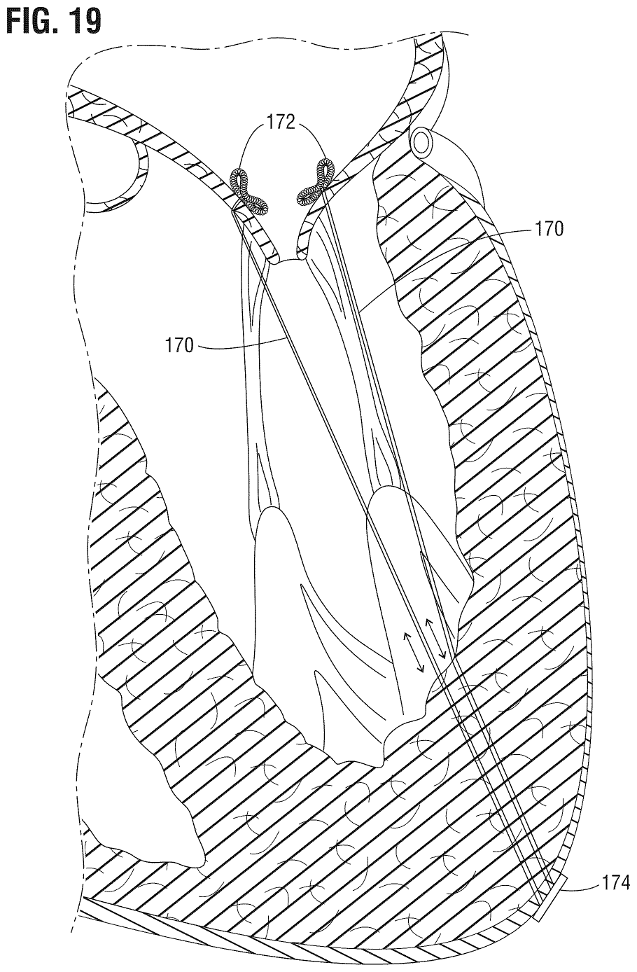

FIG. 18 is a sectional view of an ischemic left ventricle after implantation of a replacement heart valve 160 between mitral leaflets ML and remotely adjustable subvalvular structure. Specifically, a tension adjustment mechanism 162 such as described elsewhere herein may be mounted to an exterior wall of the heart valve 160. Tethers 164 connected to the tension adjustment mechanism 162 extend down to be anchored at the papillary muscles PM, such as via the use of pledgets 166, or to other subvalvular structure. After implant of the heart valve 160, tension in the tethers 164 may be adjusted remotely to pull one or more of the papillary muscles PM and thus improve the efficiency of the implanted valve. This may be done under external visualization, as described.

FIG. 19 is a sectional view of an ischemic left ventricle after implantation of remotely adjustable neochords 170. The neochords 170 attach to leaflet anchors 172 implanted on inflow sides of respective mitral leaflets ML. Neochords 170 can be secured to a tension adjustment mechanism 174 located outside the heart, such as on an exterior wall of the heart outside a pericardial layer as shown. The neochords 170 may be adjusted to be tighter or looser to improve efficiency of the implanted valve. In an alternative, a tension adjustment mechanism 174 may be provided for each neochord 170 for separate adjustment. Examples of leaflet anchors and neochord options are disclosed in WO2021195460A1, the disclosure of which is hereby incorporated by reference.

### Core Wire

The core wire can be made of 316L stainless steel wire or braided ultra-high molecular weight polyethylene (UHMWPE) and an outer polymer coating to increase lubricity. FiberWire® AR-7240 (Arthrex) and TiCron™ Blue 2 (Medtronic) are commercial examples of the latter.

### Piezo Electric Motors

The use of a piezoelectric motor to adjust the size of an annuloplasty ring realizes a number of advantages. Originally discovered in the late 1800s, piezoelectric technology utilizes the material properties of crystals and ceramics to accumulate electric charges in response to mechanical stress. This characteristic of piezo materials was leveraged in the 1960s to create vibrationally driven motors (e.g., piezoelectric motors). By exciting the piezoelectric material with a sizable electric field, the piezo material can convert excess electric charges into ultrasonic vibrations, driving the motor in various directions. Advantages over conventional brushed DC motors include an extremely compact design, minimal to no heat generation and EMI, high positional accuracy, and low part count. One important characteristic present in many piezo motor designs is a high holding force or torque (self-locking) due to the low part count and simplicity of the motor—the low part size is related to the high coulomb force of piezoelectric motors. The holding force for the present application is the force needed to keep the annuloplasty ring in the most compressed state which translates into a torque on the motor shaft. The exceptionally large frictional forces between the stator, rotor, and horn are responsible for the large holding force. Another characteristic of piezo motors is their high response rate due to the fast distortion of the crystals, leading to high linear speeds.

Recently, engineers have developed various techniques to magnify the tiny deflections of piezo actuators to create longer range motion. These devices, commonly known as piezo motors, have long been used for microscopy and nanopositioning applications in medical research labs. Medical implant applications are also being developed, and motors such as the piezo stepping drives from Physik Instrumente, with high force and precise positioning, may be suitable. Miniaturization of such motors in the body is paramount. Examples of small piezo electric motors which may be used for implant applications is disclosed in U.S. Pat. No. 7,309,943 and U.S. Patent Publication No. 2017/0229981, both to Henderson, the disclosures of which are hereby expressly incorporated herein. One tiny motor developed by New Scale Technologies Inc. of Victor, NY is less than 2.0×2.0×6.0 mm in size, weighs less than 2.0 grams, and is capable of producing up to 30 gram force linear push force with submicrometer position resolution and velocity control. The tiny motor is controlled by an application-specific integrated circuit (ASIC) driver, which can be powered by a 3V lithium battery. ASIC communication is via an Inter-Integrated Circuit (I2C) interface. Other piezo motors that may be suitable are supplied by TEKCELEO ASA of Valbonne, FR.

Some qualities of piezo motors are enhanced based upon the topology of the motor. The three main piezo motor types include resonance-motors (ultrasonic drives), piezo-walk drives, and inertia-motors (stick-slip principle) (Physik Instrumente). Each of the aforementioned piezo motor types may be utilized in the adjustable rings herein.

In the illustrated example, the motor has a holding force of at least about 9N, which prevents recoil of the ring size after adjustment from forces associated with natural cardiac cycling of the mitral valve. In other designs and in other applications, a lower holding force is suitable, for example, in a tricuspid annuloplasty ring, where a separate locking mechanism is provided, or where the coupling between the motor and the cinching element has improved mechanical advantage, for example in a worm gear design. In examples where the motor disposed adjacent the annuloplasty ring at the native annulus, the largest dimension thereof is preferably not greater than about 6 mm in any dimension. Smaller motors may also exhibit reduced holding force. Where the motor is positioned in a different location, for example, in a different location in the atrium or outside of the heart, a larger motor can be used. The illustrated example of the annuloplasty device uses a torque of about 30-50 N-mm to operate the spool. In the example illustrated in FIGS. 4A and 4B, the motor directly drives the spool, fully outputting needed the torque. In examples where the motor does not directly drive the cinching element, for example in the example illustrated in FIG. 15, a lower torque motor can be used in conjunction with a torque-enhancing mechanism, for example, a gearbox or torque multiplier, interposed between the motor and cinching element. The materials used must be biocompatible, of course, and be able to withstand years implanted in the body. Another factor to consider is the level of electronic or other interference the motor introduces. Low electronic interference reduces generation of noise which might produce artifacts that obscure the transthoracic echo (TTE) image used to visualize a procedure, potentially making it unusable. The motor should be able to increase or decrease the ring's circumference by 6 mm to provide an adequate range of adjustment. Finally, the motor must be capable of maintaining the ring size, or be self-locking, and be able to withstand a reverse rotation torque of 40 N-mm.

### Modes of Control

One consideration with the use of an implanted motor in the resizing of an annuloplasty ring, is how the motor will be controlled. A wired connection between the controller and motor is the most practical solution, though wires within the body tend to be a source of surgical complications, such as infection and increased electrical noise. Wires are also susceptible to breakage, which prevents communication altogether and can potentially lead to more serious complications. Radiofrequency (RF) telemetry is one alternative, but can require larger amounts of power, and can be difficult to efficiently transmit signals through biological tissue. The size of the antenna necessary to overcome this obstacle works against small scale implementation. Finally, galvanic coupling utilizes the conductive properties of the human body to transmit signals without the use of wires or RF telemetry. Both a receiving and transmitting electrode are coupled, and alternating current is passed through the body to relay desired signals. This can be done with the transmitter either placed on the skin, or implanted in the body along with the receiver. Any of these modes of signal transmission may be utilized to control the rings disclosed herein.

### Power

Examples of the system can be externally or internally powered, or powered by a combination of both. include an implanted power source, for example, a battery or super capacitor. The power source can have be arranged to provide sufficient power to the implanted motor, power management and control system, data communication, and any sensors as needed at any particular time. For example, in power demand for operating the sensors and storing the data therefrom can be lower than the demand for operating the internal motor. In some examples, the system has an internal power system sufficient to maintain any components that require long-term power, for example, logic, memory, sensors, and/or data communication, but not enough to operate the internal motor. Examples of such configurations prevent inadvertent adjustment of the annuloplasty ring. Additional safety provisions can be incorporated in the power management and control system that prevent unintended operation of the implanted motor.

External power sources can include an external electrical power source, which can be used to adjust the annuloplasty ring after the heart is closed and the heartbeat restarted, but before the patient's chest is closed. At this stage, the ring size can be fine tuned to reduce regurgitation, which can be monitored, for example, by echocardiography and/or using sensors incorporated in the device.

Other examples of external power sources include induction, RF (radiofrequency), and/or ultrasound, any of which can include an implanted transducer that converts the applied power into electricity, which can either charge an internal power source or itself be the sole power source permitted to operate the motor. One advantage being the sole power source for operating the motor is to eliminate or reduce accidental activation of the motor, which would only be powered, for example, when activated by a medical professional. Such an arrangement can also reduce the size/capacity of any internal power source.

Suitable internal power sources include batteries, supercapacitors, electric cells, and the like. As discussed above, the internal power source can power components that use power after the patient's chest is closed. The internal power source can also power the implanted motor, either by itself or in conjunction with an external power source.

One example of a power management module comprises of two components: an AC-DC converter and a buck converter. The staging of the power management module is illustrated in the circuit diagram of FIG. 15. The goal of the power management module is to maintain a stable voltage on both rails which supply both the motor driver and microcontroller-peripheral electronics, respectively. Because of the relatively high efficiency of the components used, cooling was not a concern.

While the foregoing is a complete description of the preferred examples, various alternatives, modifications, and equivalents may be used. Moreover, it will be obvious that certain other modifications may be practiced within the scope of the appended claims.

What is claimed is:

1. An annuloplasty ring and shape adjustment system, comprising:
an annuloplasty ring defining a continuous peripheral shape around a central orifice, and a size adjustment mechanism housed within a junction housing to adjust the peripheral shape of the annuloplasty ring and powered by a piezo-electric motor that has no ferromagnetic components.

2. The system of claim 1, wherein the annuloplasty ring is shaped for implant at a mitral annulus and has a D-shape with a rounded posterior portion opposite a more linear anterior portion, with side segments in between, and wherein the junction housing is located at a midpoint in the anterior portion.

3. The system of claim 2, wherein the posterior portion and the anterior portion rise up from the side segments such that the annuloplasty ring forms a saddle shape.

4. The system of claim 3, wherein the side segments lie in a reference plane P such that the annuloplasty ring is partially planar.

5. The system of claim 1, wherein the motor is attached to the junction housing and rotates a central shaft which engages a rotational device within the junction housing to cinch the annuloplasty ring and reduce the peripheral shape.

6. The system of claim 5, wherein the largest dimension of the motor is no greater than about 6 mm in any dimension.

7. The system of claim 5, wherein the rotational device has a shaft with a diametric passage through which an elongated flexible cinching element passes, and the cinching element passes circumferentially around the annuloplasty ring such that actuation of the motor shortens or lengthens the cinching element to respectively reduce or enlarge the peripheral shape.

8. The system of claim 5, wherein the annuloplasty ring has an elongated flexible cinching element connected to wind around the rotational device, and the cinching element passes circumferentially around the annuloplasty ring within a coiled body having opposite ends attached to the junction housing, wherein actuation of the motor shortens or lengthens the cinching element to respectively reduce or enlarge the peripheral shape.

9. The system of claim 8, wherein the cinching element is a stainless steel wire or cable.

10. The system of claim 8, wherein the coiled body defines a lumen through which a hollow compressible filler member extends, the cinching element passing through a lumen in the filler member, and the annuloplasty ring is covered with a tubular fabric cover with a sewing cuff at a peripheral outer edge.

11. The system of claim 1, wherein the motor produces a torque of about 30-50 N-mm to adjust the peripheral shape of the annuloplasty ring.

12. The system of claim 1, further including an implanted transducer that converts an externally applied power into electricity, wherein the motor is powered by the implanted transducer when energized externally.

13. The system of claim 12, further including an internal power source that provides sufficient power to a power management and control system and a data communication system but not the motor.

14. The system of claim 13, further including a sensor mounted on the annuloplasty ring and connected to the internal power source.

15. The system of claim 1, further including an internal power source that provides sufficient power to a power management and control system and a data communication system and a sensor mounted on the annuloplasty ring and connected to the power management and control system.

16. The system of claim 15, wherein the sensor is an acoustic or pressure sensor that detects regurgitation.

17. The system of claim 16, wherein the sensor provide feedback to the motor for automatic cinching if it detects regurgitation.

18. The system of claim 16, wherein the sensor cooperates with the power management and control system and a data communication system to collect and send data to an external control system for a physician to decide whether to adjust the ring.

19. The system of claim 1, wherein the motor connects to the junction housing via a flexible cable.

20. The system of claim 17, wherein the annuloplasty ring is shaped for implant at a mitral annulus and the motor is adapted to be attached outside of the left atrium and the flexible cable has a sufficient length to extend from outside the left atrial wall to the mitral annulus.
